(19) United States
(12) Patent Application Publication
(10) Pub. No.: US 2009/0197914 A1
(43) Pub. Date: Aug. 6, 2009

(54) Piperidine Derivatives, Their Process for Preparation, Their Use as Therapeutic Agents and Pharmaceutical Compositions Containing Them

(76) Inventors: Peter Cage, Leicestershire (GB); Mark Furber, Leicestershire (GB); Matthew Perry, Leicestershire (GB); Brian Springthorpe, Leicestershire (GB)

Correspondence Address: FISH & RICHARDSON P.C., P.O BOX 1022, MINNEAPOLIS, MN, 55440-1022, US

(21) Appl. No.: 12/281,127

(22) PCT Filed: Mar. 6, 2007
(86) PCT No.: PCT/SE07/00214

§ 371 Date: Aug. 28, 2008

Related U.S. Application Data
(60) Provisional application No. 60/779,903, filed on Mar. 7, 2006.

(51) Int. Cl. A61K 31/445; C07D 211/56; C07D 401/02; A61K 31/4709; A61P 37/00

(52) U.S. Cl. 514/314; 546/224; 546/168; 514/329

(57) ABSTRACT

The present invention provides a compound of a formula (I) wherein the variables are defined herein; to a process for preparing such a compound; and to the use of such a compound in the treatment of a chemokine (such as CCR3) mediated disease state.

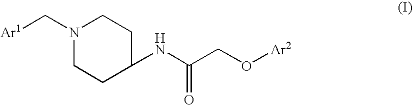

The present invention concerns novel N-benzyl-piperidine derivatives having pharmaceutical activity, to processes for preparing such derivatives, to pharmaceutical compositions comprising such derivatives and to the use of such derivatives as active therapeutic agents.

Pharmaceutically active N-benzyl-piperidine derivatives are disclosed in WO 01/14333.

The compounds of the present invention are active CCR3 antagonists and are advantageous because they have a high level of metabolic stability as shown by their intrinsic clearance. A pharmaceutically active ingredient's intrinsic clearance is a prediction of how rapidly the active ingredient would be cleared from a mammalian body, that is, it is predictor of the amount of active ingredient that would be cleared from (or metabolised by) the body in a unit of time.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CC B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

Viral infections are known to cause lung inflammation. It has been shown experimentally that the common cold increases mucosal output of eotaxin in the airways. Instillation of eotaxin into the nose can mimic some of the signs and symptoms of a common cold. (See, Greiff L et al Allergy (1999) 54(11) 1204-8 [Experimental common cold increase mucosal output of eotaxin in atopic individuals] and Kawaguchi M et al Int. Arch. Allergy Immunol. (2000) 122 S1 44 [Expression of eotaxin by normal airway epithelial cells after virus A infection].)

The present invention provides a compound of formula (I):

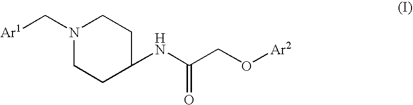

wherein:
Ar¹ is phenyl or naphthyl, either of which is optionally substituted by chloro, fluoro, methyl or CF₃;
Ar² is phenyl, naphthyl, imidazolyl, pyrazinyl, thienyl, thiazolyl, thiadiazolyl, pyridinyl, pyrimidinyl, benzimidazolyl, quinolinyl, quinazolinyl, isoquinolinyl, 5-phenylamino-1,3,4-oxadiazolyl or 3-pyridinyl-1,2,4-oxadiazolyl;
wherein Ar² is substituted by CO₂R′, tetrazolyl, (CH₂)ₘR³, CH₂CH(CH₃)R⁴, CH₂C(CH₃)₂R⁵, O(CH₂)ₖR⁶ or S(CH₂)_{q}R⁷;
and Ar² is optionally additionally substituted by one or more of halogen, hydroxy, nitro, S(O)ᵣ(C₁₋₆ alkyl), S(O)₂NH₂, S(O)₂NH(C₁₋₆ alkyl), S(O)₂N(C₁₋₆ alkyl)₂, NH₂, NH(C₁₋₆ alkyl), N(C₁₋₆ alkyl)₂, cyano, C₁₋₆ alkyl, C₁₋₆ alkoxy, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHS(O)₂(C₁₋₆ alkyl), C(O)(C₁₋₆ alkyl), CF₃ or OCF₃; wherein alkyl or alkoxy groups are optionally substituted by NR⁸R⁹;
R³, R⁴, R⁵, R⁶ and R⁷ are, independently, CO₂R* or tetrazolyl;
R⁸ and R⁹ are independently hydrogen or C₁₋₄ alkyl or together with the nitrogen to which they are attached form a ring (for example azepine, pyrrolidine, piperidine, homopiperidine, morpholine or piperazine), the latter optionally substituted on the distal nitrogen by C₁₋₄ alkyl;
R′ and R* are, independently, hydrogen, C₁₋₆ alkyl or phenyl(C₁₋₄ alkyl); wherein the phenyl is optionally substituted with halogen, hydroxy, nitro, S(O)ₜ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
or each CO₂R′ or CO₂R* is, independently, (CO₂⁻)ₚR^{p+} wherein R^{p+} is a univalent cation (for example an alkali metal cation) or two carboxylates may coordinate to a divalent cation (for example an alkaline earth metal cation);
or each tetrazolyl is, independently, (tetrazolyl^{g-})R^{g+} wherein R^{g+} is a univalent cation (for example an alkali metal cation) or two tetrazoles may coordinate to a divalent cation (for example an alkaline earth metal cation);
r and t are, independently, 0, 1 or 2;
m, k and q are, independently, 1, 2 or 3;
or a pharmaceutically acceptable salt thereof;
provided: that when Ar¹ is 3,4-dichlorophenyl then Ar² is not 3-(CO₂C₂H₅)phenyl.

Certain compounds of the present invention can exist in different isomeric forms (such as enantiomers, diastereomers, geometric isomers or tautomers). The present invention covers all such isomers and mixtures thereof in all proportions.

The compounds of the invention can be zwitterionic and all such zwitterions are within the invention.

Suitable salts include acid addition salts such as hydrochloride, dihydrochloride, hydrobromide, phosphate, sulfate, acetate, diacetate, fumarate, maleate, malonate, succinate, tartrate, citrate, oxalate, methanesulfonate, benzenesulfonate or p-toluenesulfonate.

An alkali metal cation is, for example sodium or potassium, and an alkaline earth metal cation is, for example, magnesium or calcium.

The compounds of the invention may exist as solvates (such as hydrates) and the present invention covers all such solvates.

Halogen includes fluorine, chlorine, bromine and iodine. Halogen is, for example, fluorine or chlorine.

Alkyl is straight or branched chain and is, for example, methyl, ethyl, n-propyl, iso propyl or tert-butyl.

In one particular aspect the present invention provides a compound wherein Ar¹ is phenyl optionally substituted (for example with one, two or three of the same or different) with fluorine, chlorine or methyl.

In a further aspect the present invention provides a compound wherein Ar¹ is, for example, 3-chlorophenyl, 4-chlorophenyl, 4-fluorophenyl, 3,4-difluorophenyl, 2-methyl-4-chlorophenyl, 2-methylphenyl, 3,4-dichlorophenyl, 2,4-dichloro-3-methylphenyl or 3,4-dichloro-2-methylphenyl.

In another aspect the present invention provides a compound of formula (I) wherein Ar² is substituted by CO₂R′, and optionally additionally substituted by one or more of the substituents recited above.

In a still further aspect the present invention provides a compound of formula (I) wherein Ar² is phenyl or pyridinyl substituted as recited above. In another aspect Ar² is phenyl substituted as recited above. In yet another aspect Ar² is pyridinyl substituted as recited above.

In another aspect the present invention provides a compound of formula (I) wherein Ar² is substituted by CO₂R′ or tetrazolyl (wherein R′ is hydrogen or C₁₋₄ alkyl) and is optionally additionally substituted by halogen, hydroxyl, C₁₋₄ alkyl, CF₃, C₁₋₄ alkoxy, S(O)₂NH₂, NH₂ or CH₂(morpholin-4-yl).

In yet another aspect the present invention provides a compound of formula (I) wherein Ar² is substituted by CO₂H and is optionally additionally substituted by halogen, hydroxyl, C₁₋₄ alkyl, CF₃, C₁₋₄ alkoxy, S(O)₂NH₂, NH₂ or CH₂(morpholin-4-yl).

The compounds of the present invention can be prepared as described below or by adaptation of methods described in the art (for example WO 01/14333).

A compound of formula (I) wherein CO₂R′ is an ester, can be prepared by reacting a compound of formula (II):

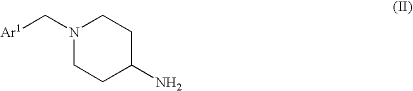

with a compound of formula (III):

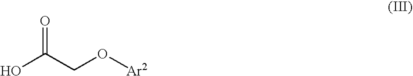

in the presence of a suitable coupling agent (such as HATU), in the presence of a suitable base (such as a tertiary amine, for example Hünig's base), in a suitable solvent (such as N-methylpyrrolidinone) at a temperature in the range −10 to 30° C.

Alternatively, a compound of formula (I) wherein CO₂R′ is an ester, can be prepared by reacting a compound of formula (IV):

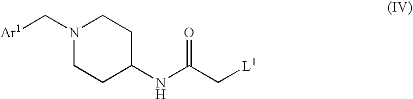

wherein L¹ is a leaving group (for example halogen, such as chloro), with a compound Ar²OH in the presence of a suitable base (for example potassium tert-butoxide) in a suitable solvent (such as NMP) at a suitable temperature (such as 0-150° C.).

For a compound of formula (I):
wherein R′ is hydrogen said compound may be converted to a compound of the invention where CO₂R′ is an ester by a standard esterification method well known in the art;
wherein CO₂R′ is an ester said compound may be converted to a compound of the invention where R is hydrogen by a standard ester hydrolysis method well known in the art; and,
wherein CO₂R′ is (CO₂⁻)ₚR^{p+} said compound can be prepared by reacting a compound wherein R is hydrogen or alkyl or substituted alkyl, with a suitable alkali metal or alkaline earth metal hydroxide.
   Such methods are described in undergraduate organic chemistry textbooks (such as Advanced Organic Chemistry by J March, 5^{th} edition M B Smith and J March, Wiley, 2001).

In the above processes it may be desirable or necessary to protect an acid group or a hydroxy or other potentially reactive group. Suitable protecting groups and details of processes for adding and removing such groups may be found in “Protective Groups in Organic Synthesis”, 3rd Edition (1999) by Greene and Wuts.

In another aspect the present invention provides processes for the preparation of compounds of formula (I).

The compounds of formula (I) have activity as pharmaceuticals, in particular as modulators of chemokine receptor (for example CCR3) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

Examples of these conditions are:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

According to a further feature of the present invention there is provided a method for treating a chemokine mediated disease state (for example a CCR3 mediated disease state) in a mammal, such as man, suffering from, or at risk of, said disease state, which comprises administering to a mammal in need of such treatment a therapeutically effective amount of a compound of the formula (I) or a pharmaceutically acceptable salt thereof.

According to yet another feature of the present invention there is provided a method for treating a sign and/or symptom of what is commonly referred to as a cold in a mammal, such as man, suffering from, or at risk of, said disease state, which comprises administering to a mammal in need of such treatment a therapeutically effective amount of a compound of the formula (I) or a pharmaceutically acceptable salt thereof.

The invention also provides a compound of the formula (I), or a pharmaceutically acceptable salt thereof, for use in therapy.

In another aspect the invention provides the use of a compound of formula (I), or a pharmaceutically acceptable salt thereof, in the manufacture of a medicament for use in therapy (for example modulating chemokine receptor activity (for example CCR3 receptor activity) or treating a sign and/or symptom of what is commonly referred to as a cold).

The invention further provides the use of a compound of formula (I), or a pharmaceutically acceptable salt thereof, in the manufacture of a medicament for use in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema; in a mammal (for example man).

In a further aspect the invention provides a compound of formula (I), or a pharmaceutically acceptable salt thereof, for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In a still further aspect a compound of formula (I), or a pharmaceutically acceptable salt thereof, is useful in the treatment of asthma.

The present invention also provides a the use of a compound of formula (I), or a pharmaceutically acceptable salt thereof, in the manufacture of a medicament for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In order to use a compound of the invention, or a pharmaceutically acceptable salt thereof, for the therapeutic treatment of a mammal, such as man, said ingredient is normally formulated in accordance with standard pharmaceutical practice as a pharmaceutical composition. Therefore in another aspect the present invention provides a pharmaceutical composition which comprises a compound of the formula (I), or a pharmaceutically acceptable salt thereof (active ingredient), and a pharmaceutically acceptable adjuvant, diluent or carrier.

In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

A compound of the invention, or a pharmaceutically acceptable salt thereof, or a pharmaceutical composition of this invention, or a combination of this invention as described below, can be administered in a standard manner for the disease condition that it is desired to treat, for example it can be administered via topical (such as to the lung and/or airways or to the skin), oral, rectal or parenteral (for example intramuscular, intravenous or intra-articular) administration.

For these purposes the compounds of this invention may be formulated by means known in the art. A suitable pharmaceutical composition of this invention is one suitable for oral administration in unit dosage form, for example a tablet or capsule which contains between 0.1 mg and 1 g of active ingredient.

Each patient may receive, for example, a dose of 0.01 mgkg⁻¹ to 100 mgkg⁻¹, for example in the range of 0.1 mgkg⁻¹ to 20 mgkg⁻¹, of the active ingredient administered, for example, 1 to 4 times per day.

The invention further relates to a combination therapy wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

The invention further relates to combination therapies wherein a compound of the invention, or a pharmaceutically acceptable salt thereof, or a pharmaceutical composition or formulation comprising a compound of the invention, or a pharmaceutically acceptable salt thereof, is administered concurrently or sequentially or as a combined preparation with one or more of the therapeutic agents listed below, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis or inflammatory bowel disease, a compound of the invention can be combined with one or more of the therapeutic agents listed below.

A non-steroidal anti-inflammatory agent (hereinafter NSAID) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

A cytokine, or agonist or antagonist of cytokine function, (including an agent which acts on a cytokine signalling pathway such as a modulator of the SOCS system) including alpha-, beta-, or gamma-interferons; insulin-like growth factor type I (IGF-1); interleukin (IL) including IL1 to 17, and interleukin antagonist or inhibitor such as anakinra; a tumour necrosis factor alpha (TNF-α) inhibitor such as anti-TNF monoclonal antibody (for example infliximab; adalimumab, and CDP-870) and TNF receptor antagonist including immunoglobulin molecule (such as etanercept) and a low-molecular-weight agent such as pentoxyfylline.

A monoclonal antibody targeting B-Lymphocytes (such as CD20 (rituximab), MRA-aIL16R and T-Lymphocytes, CTLA4-Ig, HuMax I1-15).

A modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

An inhibitor of matrix metalloprotease (MMP), such as a stromelysin, a collagenase, or a gelatinase, as well as aggrecanase; for example collagenase-1 (MMP-1), collagenase-2 (MMP-8), collagenase-3 (MMP-13), stromelysin-1 (MMP-3), stromelysin-2 (MMP-10), stromelysin-3 (MMP-11), MMP-9 or MMP-12, including an agent such as doxycycline.

A leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; a 2,6-di-tert-butylphenolhydrazone; a methoxytetrahydropyran such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, or BAY x 1005.

A receptor antagonist for leukotrienes (LT) B4, LTC4, LTD4, and LTE4 selected from the group consisting of a phenothiazin-3-1 such as L-651,392; an amidino compound such as CGS-25019c; a benzoxalamine such as ontazolast; a benzenecarboximidamide such as BIIL 284/260; or a compound such as zafirlukast, ablukast, montelukast, pranlukast, verlukast (MK-679), RG-12525, Ro-245913, iralukast (CGP 45715A), or BAY x 7195.

A phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

A histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

A proton pump inhibitor (such as omeprazole) or a gastroprotective histamine type 2 receptor antagonist.

An antagonist of the histamine type 4 receptor.

An alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

An anticholinergic agent including muscarinic receptor (M1, M2, and M3) antagonist such as atropine, hyoscine, glycopyrrrolate, ipratropium bromide, tiotropium bromide, oxitropium bromide, pirenzepine or telenzepine.

A beta-adrenoceptor agonist (including beta receptor subtypes 1-4) such as isoprenaline, salbutamol, formoterol, salmeterol, terbutaline, orciprenaline, bitolterol mesylate, pirbuterol or indacaterol, or a chiral enantiomer thereof.

A chromone, such as sodium cromoglycate or nedocromil sodium.

A glucocorticoid, such as flunisolide, triamcinolone acetonide, beclomethasone dipropionate, budesonide, fluticasone propionate, ciclesonide or mometasone furoate.

An agent that modulates a nuclear hormone receptor such as a PPAR.

An immunoglobulin (Ig) or Ig preparation or an antagonist or antibody modulating Ig function such as anti-IgE (for example omalizumab).

Another systemic or topically-applied anti-inflammatory agent, such as thalidomide or a derivative thereof, a retinoid, dithranol or calcipotriol.

An aminosalicylate or a sulfapyridine such as sulfasalazine, mesalazine, balsalazide or olsalazine; an immunomodulatory agent such as a thiopurine, or a corticosteroid such as budesonide.

An antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

A cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

A CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

An agent for the treatment of acute or chronic pain, such as a centrally or peripherally-acting analgesic (for example an opioid or derivative thereof), carbamazepine, phenyloin, sodium valproate, amitryptiline or other anti-depressant agent, paracetamol, or a non-steroidal anti-inflammatory agent.

A parenterally or topically-applied (including inhaled) local anaesthetic agent such as lignocaine or a derivative thereof.

An anti-osteoporosis agent including a hormonal agent such as raloxifene, or a biphosphonate such as alendronate.

An agent which is a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub 1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2X7; (xxvii) inhibitor of transcription factor activation such as NFkB, API, or STATS; or (xxviii) a glucocorticoid receptor modulator (such as an agonist).

A therapeutic agent for the treatment of cancer, for example:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be illustrated by the following non-limiting examples in which, unless stated otherwise:

(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-D6 (CD₃SOCD₃) or CDCl₃ as the solvent unless otherwise stated;
(ii) mass spectra (MS) were run with an electron energy of 70 electron volts in the chemical ionisation (CI) mode using a direct exposure probe; where indicated ionisation was effected by electron impact (EI) or fast atom bombardment (FAB); where values for m/z are given, generally only ions which indicate the parent mass are reported, and unless otherwise stated the mass ion quoted is the positive mass ion-(M+H)⁺;
(iii) the title and sub-title compounds of the examples and preparations were named using the index name program from Ogham and stereochemical descriptors added by hand. (See www.eyesopen.com/products/applications/ogham.html);
(iv) unless stated otherwise, reverse phase HPLC was conducted using a “Symmetry”, “NovaPak” or “Xterra” reverse phase silica column, all available from Waters Corp.;
(v) for analytical HPLC the following conditions were used:

Reverse phase analytical HPLC (Hewlett Packard Series 1100) using Waters “Symmetry” C8 column 3.5 μm; 4.6×50 mm column using 0.1% ammonium acetate/acetonitrile gradients at 2 mL/min given as % aqueous

STANDARD 75% to 5% over 3 min
FAST 45% to 5% over 2.5 min

### MEDIUM FAST 65% to 5% in 2.5 min

### SLOW 95% to 50% in 2.5 min

SUPERSLOW 100% to 80% in 2.5 min; and
(vi) the following abbreviations are used:

| | |
|---|---|
| DMF | N,N-Dimethylformamide |
| HPLC | High performance liquid chromatography |
| RPHPLC | Reverse phase high performance liquid chromatography |
| HATU | O-(7-Benzotriazol-1-yl)-N,N,N′,N′-tetramethyluronium |
| | hexafluorophosphate |
| THF | Tetrahydrofuran |
| DCM | Dichloromethane |
| d | Day(s) |
| h | Hour(s) |
| min | Minute(s) |
| RT | Room Temperature |

### Intermediate 1

### {[2-(Methoxycarbonyl)quinolin-6-yl]oxy}acetic Acid

### a) Methyl 6-(2-tert-butoxy-2-oxoethoxy)quinoline-2-carboxylate

Methyl 6-hydroxylquinoline-2-carboxylate (0.5 g) and cesium carbonate (1.2 g) were stirred in dry DMF (5 mL). tert-Butylbromoacetate (0.4 mL) was added and the mixture was stirred for 18 h. The mixture was diluted with ethyl acetate and was washed with sodium bicarbonate solution, water and then brine. The solution was dried and evaporated to afford the subtitle compound (0.75 g).

¹NMR [symbol] _{(CDCL3)} 1.50 (9H, s), 4.07 (3H, s), 4.67 (2H, s), 7.04 (1H, d), 7.51 (1H, dd), 8.16 (2H, dd), 8.23 (1H, d).

### b) {[2-(Methoxycarbonyl)quinolin-6-yl]oxy}Acetic Acid

The product from part a) (0.75 g) was stirred in a mixture of dichloromethane (3 mL) and trifluoroacetic acid (5 μL) at 0° C. for 1.5 h and then at room temperature for 1 h. The solvent was evaporated and the residue was partitioned between ethyl acetate and water. The aqueous layer was neutralised with sodium bicarbonate and then acidified with acetic acid. The mixture was extracted with ethyl acetate. The extracts were washed with brine and then dried and evaporated to leave the title compound as a yellow solid (0.73 g).

¹H NMR [symbol] _{(DMSO)} 3.94 (3H, s), 4.89 (2H, s), 7.45 (1H, d), 7.55 (1H, dd), 8.02 (2H, m), 8.43 (1H, d).

### Intermediate 2

### 2-Amino-6-carboxymethoxy-5-fluoro-nicotinic Acid Methyl Ester

### a) 2-Chloro-5-fluoro-6-[(4-methylphenyl)thio]nicotinic Acid

Lithium hydroxide monohydrdrate (1.8 g) was added to a solution of ethyl 2-chloro-5-fluoro-6-[(4-methylphenyl)thio]nicotinate (7.0 g) in THF (100 mL). Water (20 mL) was added and the solution stirred vigorously for 18 h. The mixture was diluted with water (400 mL) and washed with ether. The aqueous was acidified with acetic acid and extracted with ether. The ether was dried and evaporated to give the subtitle compound (6.0 g).

¹H NMR δ_{(DMSO)} 2.37 (3H, s), 7.31 (2H, d), 7.47 (2H, d), 8.12 (1H, d).

### b) 2-amino-5-fluoro-6-[(4-methylphenyl)thio]nicotinic Acid

The product from part a) (1 g) and aqueous ammonia (density 0.880) (20 mL) were heated in a sealed tube at 140° C. for 5 h. The cooled mixture was evaporated and the residue was coevaporated with methanol (×3) then dissolved in methanol and acidified with acetic acid. The mixture was evaporated and coevaporated with methanol (×2) then toluene (×2). Final drying under high vacuum gave the subtitle compound (0.95 g).

¹H NMR δ_{(DMSO)} 2.33 (3H, s), 7.24 (3H, m), 7.38 (2H, d).

### c) Methyl 2-amino-5-fluoro-6-[(4-methylphenyl)thio]nicotinate

The product from part b) (0.95 g) was stirred in thionyl chloride (5 mL) and heated under reflux for 1 h. The solvent was evaporated and the residue dissolved in ice cooled methanol (10 mL). The mixture was evaporated and the residue mixes with saturated sodium bicarbonate solution and extracted with ethyl acetate. The extracts were washed with brine then dried and evaporated. Purification by flash chromatography (ethyl acetate/isohexane 9:1) gave the subtitle compound (0.3 g).

¹H NMR δ_{(CDCl3)} 2.39 (3H, s), 3.85 (3H, s), 7.21 (2H, d), 7.42 (2H, d), 7.67 (1H, d).

### d) Methyl 2-amino-5-fluoro-6-[(4-methylphenyl)sulfonyl]nicotinate

The product from part c) (0.74 g) was stirred in dichloromethane (5 mL) and m-chloroperoxybenzoic acid (1.13 g of 77%) was added. The mixture was stirred for 2 h then was washed with sodium bicarbonate solution followed by sodium metabisulfite solution then brine. The solution was dried and evaporated and the residue purified by flash chromatography (ethyl acetate/dichloromethane (1:1) to give the subtitle compound (0.43 g).

¹H NMR δ_{(CDCl3)} 2.44 (3H, s), 3.90 (3H, s), 7.35 (2H, m), 7.94 (3H, m). MS 325 [M+H]⁺ (APCI+).

e) Methyl glycolate (0.077 mL) was added to a solution of potassium tert-butoxide (0.112 g) in dry DMF (2 mL) and the mixture stirred for 10 minutes. A solution of the product from part b) (0.324 g) in dry DMF (2 mL) was added and the resulting solution was stirred at 80° C. for 2 h. Further methyl glycolate (0.015 mL) was added and the mixture heated at 100° C. for 3 h. The mixture was evaporated and the residue mixed with water and acidified with acetic acid. The mixture was extracted with ethyl acetate. The extracts were washed with brine then dried and evaporated. The residue was purified by flash chromatography (ethyl acetate/isohexane 1:9) to give the subtitle compound as a colourless solid (0.092 g)

¹H NMR δ_{(CDCl3)} 3.78 (3H, s), 3.85 (3H, s), 4.92 (2H, s), 7.80 (1H, d).

f) The compound from part e) (0.143 g) was stirred in THF (20 mL) and water (5 mL) with lithium hydroxide monohydrate (0.023 g) for 16 h. The mixture was diluted with water (100 mL) and acidified with acetic acid. The mixture was extracted with ethyl acetate and the extracts washed with brine, dried and evaporated to give the title compound as a solid (0.113 g)

¹H NMR δ_{(DMSO)} 3.77 (3H, s), 4.92 (2H, s), 7.20 (2H, bs), 7.83 (1H, d). MS 245 [M+H] (APCI+).

### Intermediate 3

### 2-Chloro-N-[1-(3,4-dichloro-2-methyl-benzyl)-piperidin-4-yl]-acetamide

### a) 3,4-Dichloro-2-methyl-benzaldehyde

n-Butyl lithium (1.6 M in hexanes, 1.29 mL) was added dropwise to a solution of N,N,N′-trimethylthylene-diamine (0.27 mL) in tetrahydrofuran (5.3 mL) at −78° C. After stirring for 15 min 3,4-dichlorobenzaldehyde (0.350 g) was added and the mixture was stirred for a further 15 min. n-Butyllithium (1.6 M in hexanes, 3.75 mL) was added and the temperature was maintained for 5 h then methyl iodide (0.75 mL) was added and the mixture was allowed to stand at −20° C. overnight. The mixture was poured into 10% hydrochloric acid and was extracted with diethyl ether, the organics were combined, washed with saturated brine, dried over anhydrous magnesium sulfate, and then evaporated. Purificationby flash chromatography (ethyl acetate/iso-hexane 0.1:99.9) afforded the subtitle compound (0.179 g).

### b) [1-(3,4-Dichloro-2-methyl-benzyl)-piperidin-4-yl]-carbamic Acid Tert-Butyl Ester

To a stirred solution of triethylamine (0.13 mL) and piperidin-4-yl-carbamic acid tert-butyl ester hydrochloride (0.189 g) in tetrahydrofuran (1 mL) was added the product from part a) (0.179 g), sodium triacetoxyborohydride (0.302 g) and acetic acid (0.08 mL). The mixture was stirred overnight at room temperature, then partitioned between saturated sodium bicarbonate solution and dichloromethane. The aqueous phase was extracted with dichloromethane, the organics were combined, evaporated, and the residue was purified by flash chromatography (ethyl acetate/iso-hexane 1:4) to give the subtitle compound (0.087 g).

MS 373/375 [M+H]⁺ (APCI+)

### c) 1-(3,4-Dichloro-2-methyl-benzyl)-piperidin-4-ylamine

A solution of the product from part b) (0.087 g) and trifluoroacetic acid (0.16 mL) in dichloromethane (0.72 mL) was stirred at room temperature overnight. Purification by SCX chromatography (methanol then 7 N NH₃ in methanol) afforded the subtitle compound (0.033 g).

### d) 2-Chloro-N-[1-(3,4-dichloro-2-methyl-benzyl)-piperidin-4-yl]-acetamide

A solution of the product from part c) (0.215 g) in dichloromethane (2.2 mL) was cooled below 0° C. Triethylamine (0.14 mL) was added and then chloroacetyl chloride (0.069 mL) in dichloromethane (0.56 mL) was added dropwise. After stirring for 1 h, the mixture was poured onto water and the layers were separated. The aqueous phase was extracted with dichloromethane, and the combined extracts were washed with sodium bicarbonate solution, dried over anhydrous sodium sulfate, filtered and evaporated. The resulting brown solid was purified by flash chromatography (ethyl acetate:iso-hexane 1:1 then ethyl acetate) to give the title compound (0.069 g).

MS 351/353 [M+H⁺] (APCI+)

¹H NMR δ_{(CD3OD)} δ1.65-1.47 (m, 2H), 1.86 (d, 2H), 2.27-2.12 (m, 2H), 2.49 (s, 3H), 2.86 (d, 2H), 3.52 (s, 2H), 3.80-3.65 (m, 1H), 4.01 (s, 2H), 7.21 (d, 1H) and 7.33 (d, 1H).

### EXAMPLE 1

### 4-(2-{[1-(3,4-Dichlorobenzyl)piperidin-4-yl]amino}-2-oxoethoxy)benzoic Acid

### a) Methyl 4-(2-{[1-(3,4-dichlorobenzyl)piperidin-4-yl]amino}-2-oxoethoxy)benzoate

[4-(Methoxycarbonyl)phenoxy]acetic acid (0.081 g), diisopropylethylamine (0.135 mL) and (3,4-Dichlorobenzyl)piperidin-4-amine (0.1 g) were stirred in dry NMP (1 mL). HATU (0.161 g) was added and stirring was continued for 18 h. The solvent was evaporated and the mixture was purified by flash chromatography (dichloromethane then ethyl acetate/dichloromethane 1:1) to give the subtitle compound as an oil (0.175 g).

MS 451/453 [M+H] (APCI+).

### b) 4-(2-{[1-(3,4-Dichlorobenzyl)piperidin-4-yl]amino}-2-oxoethoxy)benzoic Acid

The product from part a) (0.35 g) and lithium hydroxide monohydrate (0.065 g) were stirred in water (10 mL) and THF (10 mL) at room temperature for 2 days. The mixture was acidified with acetic acid and evaporated. Purification by reverse phase HPLC gave the title compound as a colourless solid (0.137 g).

MS 437/439 [M+H]⁺ (APCI+)

¹H NMR δ_{(CD3OD+NaOD)} 1.67 (2H, m), 1.89 (2H, m), 2.29 (2H, m), 2.92 (2H, m), 3.61 (2H, s), 3.84 (1H, m), 4.49 (2H, s), 7.05 (2H, d), 7.29 (1H, d), 7.50 (1H, d), 7.56 (1H, s), 8.00 (2H, d).

Also Prepared by this route:

| | | | MS | |
|---|---|---|---|---|
| | | | [M + H]⁺ | |
| Example | Name | Prepared from | (APCI+) | ¹H NMR |
| 2 | 6-(2-{[1-(3,4- | {[6- | 486/488 | δ_{CD3OD+NaOD)} 1.64 (2H, |
| | Dichlorobenzyl)piperidin- | (Methoxycarbonyl)- | | m), 1.85 (2H, m), |
| | 4- | 2- | | 2.15 (2H, m), 2.79 (2H, m), |
| | yl]amino}-2- | naphthyl]oxy}acetic | | 3.46 (2H, s), 3.82 (1H, |
| | oxoethoxy)-2- | acid | | m), 4.63 (2H, s), |
| | naphthoic acid | | | 7.23 (2H, m), 7.28 (1H, dd), |
| | | | | 7.45 (1H, d), 7.50 (1H, |
| | | | | d), 7.71 (1H, d), |
| | | | | 7.87 (1H, d), 8.01 (1H, dd), |
| | | | | 8.40 (1H, s). |
| 3 | 6-(2-{[1-(3,4- | {[2- | 488/490 | δ_{(CD3OD+NaOD)} 1.63 (2H, |
| | Dichlorobenzyl)piperidin- | (Methoxycarbonyl)quinolin- | | m), 1.85 (2H, m), |
| | 4- | 6- | | 2.14 (2H, m), 2.82 (2H, m), |
| | yl]amino}-2- | yl]oxy}acetic acid | | 3.47 (2H, s), 3.81 (1H, |
| | oxoethoxy)quinoline- | | | m), 4.66 (2H, s), |
| | 2-carboxylic | | | 7.23 (1H, dd), 7.29 (1H, d), |
| | acid | | | 7.45 (1H, d), 7.50 (1H, |
| | | | | d), 7.54 (1H, dd), |
| | | | | 8.10 (1H, d), 8.13 (1H, d), |
| | | | | 8.24 (1H, d). |
| 4 | 2-Amino-6-{[1- | 2-Amino-6- | 485/487 | δ_{(CD3OD+NaOD)} 1.52 (2H, |
| | (3,4-dichloro-2- | carboxymethoxy- | | m), 1.79 (2H, m), |
| | methyl-benzyl)- | 5-fluoro-nicotinic | | 2.14 (2H, m), 2.43 (3H, s), |
| | piperidin-4- | acid methyl ester | | 2.74 (2H, m), 3.45 (2H, |
| | ylcarbamoyl]- | | | s), 3.74 (1H, m), |
| | methoxy}-5- | | | 4.73 (2H, s), 7.15 (1H, d), |
| | fluoro-nicotinic | | | 7.28 (1H, d), 7.82 (1H, |
| | acid | | | d). |
| 5 | 2-Amino-6-{[1- | 2-Amino-6- | 471/473 | δ_{(CD3OD+NaOD)} 1.59 (2H, |
| | (3,4- | carboxymethoxy- | | m), 1.85 (2H, m), |
| | dichlorobenzyl)- | 5-fluoro-nicotinic | | 2.26 (2H, m), 2.87 (2H, m), |
| | piperidin-4- | acid methyl ester | | 3.57 (2H, m), 3.76 (1H, |
| | ylcarbamoyl]- | | | m), 4.77 (2H, s), |
| | methoxy}-5- | | | 7.25 (1H, dd), 7.46 (1H, d), |
| | fluoro-nicotinic | | | 7.52 (1H, d) 7.79 (1H, d). |
| | acid | | | |

### EXAMPLE 6

### [4-(2-{[1-(3,4-Dichlorobenzyl)piperidin-4-yl]amino}-2-oxoethoxy)phenyl]acetic Acid

### 5a) Methyl [4-(2-{[1-(3,4-dichlorobenzyl)piperidin-4-yl]amino}-2-oxoethoxy)phenyl]acetate

Methyl (4-hydroxyphenyl)acetate (0.149 g) was stirred in N-methylpyrrolidin-2-one (10 mL) under nitrogen. Potassium tert-butoxide (0.094 g) was added. After 15 minutes 2-chloro-N-[1-(3,4-dichlorobenzyl)piperidin-4-yl]acetamide (0.2 g) was added and the mixture was heated at 60° C. for 17 hours. The mixture was then cooled and poured into water. The aqueous mixture was extracted with ethyl acetate, the extracts were washed with brine, dried and evaporated. Purification of the residue by flash chromatography, (ethyl acetate) afforded the subtitle compound (0.166 g).

MS 465/467 [M+H]⁺ (ESI+), RT (standard gradient) 2.36 minutes.

### b) [4-(2-{[1-(3,4-Dichlorobenzyl)piperidin-4-yl]amino}-2-oxoethoxy)phenyl]acetic Acid

The product from part a) (0.166 g) was stirred in THF (1 mL) and water (7 mL) with lithium hydroxide monohydrate (0.121 g) overnight. The solution was acidified with acetic acid and evaporated. Purification of the residue by reverse phase HPLC afforded the title compound as a colourless solid (0.120 g).

MS 451/453 [M+H]⁺ (APCI+)

¹H NMR δ_{(DMSO)} 1.51 (2H, qd), 1.69 (2H, d), 2.03 (2H, t), 2.73 (2H, d), 3.46 (4H, d), 3.57-3.69 (1H, m), 4.42 (2H, s), 6.88 (2H, d), 7.16 (2H, d), 7.29 (1H, dd), 7.54 (1H, d), 7.58 (1H, d), 7.92 (1H, d).

Prepared by this route:

| | | MS | |
|---|---|---|---|
| | | [M + H]⁺ | |
| Example | Name | (APCI+) | ¹H NMR |
| 7 | [3-(2-{[1-(3,4- | 451/453 | δ_{(DMSO)} 1.51 (2H, dd), 1.69 (2H, |
| | Dichlorobenzyl)piperidin-4- | | d), 2.03 (2H, t), 2.73 (2H, d), |
| | yl]amino}-2- | | 3.47 (4H, d), 3.58-3.69 (1H, |
| | oxoethoxy)phenyl]acetic acid | | m), 4.42 (2H, s), |
| | | | 6.79-6.88 (3H, m), 7.21 (1H, t), 7.29 (1H, |
| | | | dd), 7.54 (1H, d), 7.58 (1H, d), |
| | | | 7.94 (1H, d). |
| 8 | [4-(2-{[1-(3,4- | 467/469 | δ_{(DMSO)} 1.45-1.58 (2H, m), |
| | Dichlorobenzyl)piperidin-4- | | 1.68 (2H, d), 2.02 (2H, t), 2.73 (2H, |
| | yl]amino}-2- | | d), 3.46 (2H, s), 3.55-3.71 (1H, |
| | oxoethoxy)phenoxy]acetic acid | | m), 4.36 (4H, s), 6.82 (4H, ddd), |
| | | | 7.29 (1H, dd), 7.54 (1H, d), |
| | | | 7.58 (1H, d), 7.88 (1H, d). |
| 9 | 3-{[1-(3,4-Dichloro-benzyl)- | 437/439 | δ_{(CD3OD+NaOD)} 1.60-1.78 (2H, |
| | piperidin-4-ylcarbamoyl]- | | m), 1.85-1.97 (2H, m), |
| | methoxy}-benzoic acid | | 2.32-2.45 (2H, m), 2.92-3.03 (2H, |
| | | | m), 3.69 (2H, s), |
| | | | 3.79-3.93 (1H, m), 4.57 (2H, s), |
| | | | 7.15-7.22 (1H, m), 7.27-7.34 (1H, |
| | | | m), 7.36-7.43 (1H, m), |
| | | | 7.48-7.55 (1H, m), 7.56-7.63 (2H, |
| | | | m), 7.62-7.69 (1H, m) |
| 10 | 4-{[1-(3,4-Dichloro-benzyl)- | 455/457 | δ_{(CD3OD+NaOD)} 1.59-1.71 (2H, |
| | piperidin-4-ylcarbamoyl]- | | m), 1.87-1.95 (2H, m), |
| | methoxy}-3-fluoro-benzoic | | 2.32-2.41 (2H, m), 2.92-3.00 (2H, |
| | acid | | m), 3.66 (2H, s), |
| | | | 3.79-3.88 (1H, m), 4.64 (2H, s), 7.09 (1H, |
| | | | t), 7.29 (1H, dd), 7.50 (1H, d), |
| | | | 7.56 (1H, d), 7.72 (1H, dd), |
| | | | 7.75-7.80 (1H, m) |

### EXAMPLE 11

### Human Eosinophil Chemotaxis

Human eosinophils are isolated from EDTA anticoagulated peripheral blood as previously described (Hansel et al., J. Immunol. Methods, 1991, 145, 105-110). The cells are resuspended at 10×10⁶ mL¹ in RPMI containing 200 IU/mL penicillin, 200 μg/mL streptomycin sulfate and supplemented with 10% HIFCS, at room temperature.

Eosinophils (700 μl) ae pre-incubated for 15 mins at 37° C. with 7 μl of either vehicle or compound (100× required final concentration in 10% DMSO). A chemotaxis plate (ChemoTx, 3 μm pore, Neuroprobe) can be loaded by adding 28 μl of a concentration of eotaxin 0.1 to 100 nM (a selective CCR3 agonist over this concentration range) containing a concentration of a compound according to the Examples or solvent to the lower wells of the chemotaxis plate. The filter is then placed over the wells and 25 μl of eosinophil suspension is added to the top of the filter. The plate is incubated for 1 hr at 37° C. in a humidified incubator with a 95% air/5% CO₂ atmosphere to allow chemotaxis.

The medium, containing cells that had not migrated, is carefully aspirated from above the filter and discarded. The filter is then washed once with phosphate buffered saline (PBS) containing 5 mM EDTA to remove any adherent cells. Cells that have migrated through the filter are pelleted by centrifugation (300×g for 5 mins at room temperature) and the filter removed and the supernatant transferred to each well of a 96-well plate (Costar). The pelleted cells are lysed by the addition of 28 μl of PBS containing 0.5% Triton ×100 followed by two cycles of freeze/thawing. The cell lysate is then added to the supernatant. The number of eosinophils migrating can be quantified according to the method of Strath et al., J. Immunol. Methods, 1985, 83, 209 by measuring eosinophil peroxidase activity in the supernatant.

### EXAMPLE 12

### Eotaxin-2-Induced Shape Change in Eosinophils in Human Blood In Vitro

See for example, Differential regulation of eosinophil chemokine signaling via CCR3 and non-CCR3 pathways. Sabroe I, Hartnell A, Jopling L A, Bel S, Ponath P D, Pease J E, Collins P D, Williams T J. J Immunol. 1999 Mar. 1; 162(5):2946-55.

Human blood, collected by venous puncture into 9 mL lithium-heparin tubes, was incubated with the CCR3 agonist eotaxin-2 in the presence of vehicle (0.1% (v/v) DMSO) or test compound for 4 min at 37° C. in a deep, 96-square-well plate. The blood was fixed with Optilyse B (100 μL) at room temperature for 10 min and then the red blood cells were lysed with distilled water (1 mL) for 60 min at room temperature.

The plate was centrifuged at room temperature for 5 min at 300 g. The pellet was re-suspended in assay buffer (PBS without CaCl₂ and MgCl₂, containing HEPES (10 mM), Glucose (10 mM) and 0.1% (w/v) BSA, pH 7.4)) and the samples were analysed using flow cytometry (FC500, Beckman Coulter). The high autofluorescence of eosinophils allowed them to be identified as a discrete population from the other blood cell types. Eosinophil shape was monitored as the refractive index of the eosinophil population as determined using the forward scatter signal in flow cytometry.

Eotaxin-2 induced a concentration-dependent change in the forward scatter of eosinophils and these data were used to construct a concentration effect curve (E/[A] curve). The rightward displacement of the eotaxin-2 E/[A] curve in the presence of a CCR3 antagonist was used to estimate a pA₂ value in blood using the following equation:

Single pA₂=−log₁₀([B]/(r−1))

where r is the ratio of the concentrations required for half maximal effects of eotaxin-2 in the absence and presence of antagonist ([A]₅₀ for eotaxin-2 in the presence of antagonist divided by [A]₅₀ for control eotaxin-2 curve) and [B] is the molar concentration of antagonist.

### EXAMPLE 13

### Determination of Compound Affinity at Human Recombinant CCR3 Receptors Assessed By Competition of [³H]-4-(2,4-dichloro-3-methylphenoxy)-1′-[4-(methylsulfonyl)benzoyl]-1,4′-bipiperidine for CHO-K1 Cell Membranes In Vitro

Membranes, prepared from CHO-K1 cells stably expressing recombinant human CCR3, suspended in assay buffer (50 mM Tris-Base, pH 7.4; containing sodium chloride (100 mM) and magnesium chloride (2 mM)) were incubated in the presence of 2 nM [³H]-4-(2,4-dichloro-3-methylphenoxy)-1′-[4-(methylsulfonyl)benzoyl]-1,4′-bipiperidine, along with vehicle (1% (v/v) DMSO), 4-(4-chloro-3-methylphenoxy)-1′-[2-(methylsulfonyl)benzoyl]-1,4′-bipiperidine (to define non-specific binding) or test compound for 2 h at 37° C. in round bottomed 96-well plates. The plates were then filtered onto GF/B filter plates, pre-soaked for 1 hour in plate-coating solution (0.3% (w/v) polyethylenimine, 0.2% (w/v) BSA in de-ionised water), using a 96-well plate Tomtec cell harvester. Four washes (250 μL) with wash buffer (50 mM Tris-Base, pH 7.4 containing sodium chloride (500 mM) and magnesium chloride (2 mM)) were performed at 4° C. to remove unbound radioactivity. Plates were dried and MicroScint-O (50 μL) was added to each well. The plates were sealed (TopSeal A) and filter-bound radioactivity was measured with a scintillation counter (TopCount, Packard BioScience) using a 1 minute counting protocol.

Specific binding was determined from values of the control wells minus the values for the NSB wells for each assay plate. PIC₅₀ values were calculated using a four parameter logistic fit (where pIC₅₀ is defined as the negative logarithm of the concentration of compound required for 50% reduction in specific [³H]-4-(2,4-dichloro-3-methylphenoxy)-1′-[4-(methylsulfonyl)benzoyl]-1,4′-bipiperidine binding). Data were presented as mean pKi values (calculated by applying a Cheng-Prussof correction to pIC₅₀ values) from a minimum of 2 separate experiments. Results are shown in Table I below.

### EXAMPLE 14

### Determination of Intrinsic Clearance with Human Liver Microsomes

Frozen human liver microsomes (BD Gentest, Oxford) were defrosted and were then diluted with 0.1 M pH 7.4 phosphate buffer at 4° C. to 1 mg protein/ml. 0.45 mL aliquots of the microsome suspension were dispensed into flat-bottomed vials (1 per compound) and were allowed to come to room temperature (5 min). During the warming time, 5 μL of solution of each test compound (typically 100 μM in DMSO) was dispensed into separate vials resulting in a final DMSO concentration of 1%. 50 μL of a 10 mM solution of NADPH in phosphate buffer (0.1 M pH 7.4, 37° C.) was added to each vial to initiate metabolism.

50 μL Aliquots of the mixtures were removed at measured intervals and were immediately quenched by addition to 100 μL of methanol cooled in ice. The quenched samples were kept cold (at −20° C. or less) for at least 1 h and were then centrifuged to remove protein.

The supernatant solution was analysed using quantitative LCMS for the presence of test compound. From the concentrations of test compound at different time points a T_{1/2} may be calculated which may be converted to an intrinsic clearance using the equation: CLᵢₙₜ=ln 2/T_{1/2}. A result is shown in Table I below.

TABLE I

| | | CLint human microsomes |
|---|---|---|
| Example | CCR3 pKi | (mL/min/kg) |
| 4 | 8.5 | <3 |

1. A compound of formula (I): 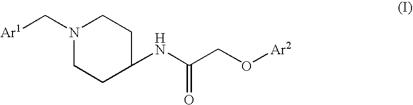
wherein:
Ar¹ is phenyl or naphthyl, either of which is optionally substituted by chloro, fluoro, methyl or CF₃;
Ar² is phenyl, naphthyl, imidazolyl, pyrazinyl, thienyl, thiazolyl, thiadiazolyl, pyridinyl, pyrimidinyl, benzimidazolyl, quinolinyl, quinazolinyl, isoquinolinyl, 5-phenylamino-1,3,4-oxadiazolyl or 3-pyridinyl-1,2,4-oxadiazolyl;
wherein Ar² is substituted by CO₂R′, tetrazolyl, (CH₂)ₘR³, CH₂CH(CH₃)R⁴, CH₂C(CH₃)₂R⁵, O(CH₂)ₖR⁶ or S(CH₂)_{q}R⁷;
and Ar² is optionally additionally substituted by one or more of halogen, hydroxy, nitro, S(O)ᵣ(C₁₋₆ alkyl), S(O)₂NH₂, S(O)₂NH(C₁₋₆ alkyl), S(O)₂N(C₁₋₆ alkyl)₂, NH₂, NH(C₁₋₆ alkyl), N(C₁₋₆ alkyl)₂, cyano, C₁₋₆ alkyl, C₁₋₆ alkoxy, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHS(O)₂(C₁₋₆ alkyl), C(O)(C₁₋₆ alkyl), CF₃ or OCF₃; wherein alkyl or alkoxy groups are optionally substituted by NR⁸R⁹;
R³, R⁴, R⁵, R⁶ and R⁷ are, independently, CO₂R* or tetrazolyl;
R⁸ and R⁹ are independently hydrogen or C₁₋₄ alkyl or together with the nitrogen to which they are attached form a ring (for example azepine, pyrrolidine, piperidine, homopiperidine, morpholine or piperazine), the latter optionally substituted on the distal nitrogen by C₁₋₄ alkyl;
R′ and R* are, independently, hydrogen, C₁₋₆ alkyl or phenyl(C₁₋₄ alkyl); wherein the phenyl is optionally substituted with halogen, hydroxy, nitro, S(O)ₜ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
or each CO₂R′ or CO₂R* is, independently, (CO₂⁻)ₚR^{p+} wherein R^{p+} is a univalent cation (for example an alkali metal cation) or two carboxylates may coordinate to a divalent cation (for example an alkaline earth metal cation);
or each tetrazolyl is, independently, (tetrazolyl^{g-})R^{g+} wherein R^{g+} is a univalent cation (for example an alkali metal cation) or two tetrazoles may coordinate to a divalent cation (for example an alkaline earth metal cation);
r and t are, independently, 0, 1 or 2;
m, k and q are, independently, 1, 2 or 3;
or a pharmaceutically acceptable salt thereof;
provided: that when Ar¹ is 3,4-dichlorophenyl then Ar² is not 3-(CO₂C₂H₅)phenyl.

2. A compound of formula (I) as claimed in claim 1 wherein Ar¹ is phenyl optionally substituted with fluorine, chlorine or methyl.

3. A compound of formula (I) as claimed in claim 1 wherein Ar¹ is 3-chlorophenyl, 4-chlorophenyl, 4-fluorophenyl, 3,4-difluorophenyl, 2-methyl-4-chlorophenyl, 2-methylphenyl, 3,4-dichlorophenyl, 2,4-dichloro-3-methylphenyl or 3,4-dichloro-2-methylphenyl.

4. A compound of formula (I) as claimed in claim 1, 2 or 3 wherein Ar² is phenyl or pyridinyl substituted as recited in claim 1.

5. A compound of formula (I) as claimed in claim 1 wherein Ar² is phenyl substituted as recited in claim 1.

6. A compound of formula (I) as claimed in claim 1, 2 or 3 wherein Ar² is substituted by CO₂R′ or tetrazolyl (wherein R′ is hydrogen or C₁₋₄ alkyl) and is optionally additionally substituted by halogen, hydroxyl, C₁₋₄ alkyl, CF₃, C₁₋₄ alkoxy, S(O)₂NH₂, NH₂ or CH₂(morpholin-4-yl).

7. A compound of formula (I) as claimed in claim 1 wherein Ar² is substituted by CO₂H and is optionally additionally substituted by halogen, hydroxyl, C₁₋₄ alkyl, CF₃, C₁₋₄ alkoxy, S(O)₂NH₂, NH₂ or CH₂(morpholin-4-yl).

8. A process for preparing a compound of formula (I) as claimed in claim 1, the process comprising:
a) when CO₂R′ is an ester, reacting a compound of formula (II): 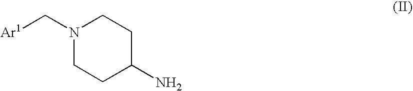
with a compound of formula (III): 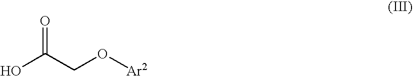
in the presence of a suitable coupling agent, in the presence of a suitable base, in a suitable solvent, at a temperature in the range −10 to 30° C.; or,
b) when CO₂R′ is an ester, reacting a compound of formula (IV): 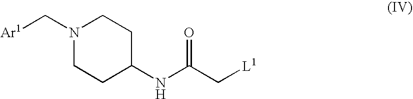
wherein L¹ is a leaving group, with a compound Ar²OH in the presence of a suitable base in a suitable solvent at a suitable temperature;
c) when R′ is hydrogen said compound may be converted to a compound of the invention where CO₂R′ is an ester by a standard esterification method well known in the art;
d) when CO₂R′ is an ester said compound may be converted to a compound of the invention where R is hydrogen by a standard ester hydrolysis method well known in the art; or,
e) when CO₂R′ is (CO₂⁻)ₚR^{p+} said compound can be prepared by reacting a compound wherein R is hydrogen or alkyl or substituted alkyl, with a suitable alkali metal or alkaline earth metal hydroxide.

9. A pharmaceutical composition which comprises a compound of the formula (I), or a pharmaceutically acceptable salt thereof as claimed in claim 1, and a pharmaceutically acceptable adjuvant, diluent or carrier.

10. A compound of the formula (I), or a pharmaceutically acceptable salt thereof as claimed in claim 1, for use in therapy.

11. A compound of formula (I), or a pharmaceutically acceptable salt thereof as claimed in claim 1, in the manufacture of a medicament for use in therapy.

12. A method of treating a chemokine mediated disease state in a mammal suffering from, or at risk of, said disease, which comprises administering to a mammal in need of such treatment a therapeutically effective amount of a compound of formula (I), or a pharmaceutically acceptable salt thereof as claimed in claim 1.
